(19) United States
(12) Patent Application Publication
(10) Pub. No.: US 2015/0017705 A1
(43) Pub. Date: Jan. 15, 2015

(54) Method and a system for mass-cultivating microalgae with enhanced photosynthetic efficiency

(71) Applicant: TET SHIN HO, W.P Kuala Lumpur (MY)

(72) Inventor: Tet Shin Ho, Kuala Lumpur (MY)

(21) Appl. No.: 14/366,096

(22) PCT Filed: Nov. 29, 2012
(86) PCT No.: PCT/MY2012/000290

§ 371 Date: Jun. 17, 2014

(30) Foreign Application Priority Data
Dec. 29, 2011 (MY) PI 2011700209

(51) Int. Cl. C12M 1/34; C12N 1/12

(52) U.S. Cl. CPC C12M 41/46; C12N 1/12
USPC 435/257.1; 435/286.5

(57) ABSTRACT

A method for cultivating photosynthetic microorganisms with optimal photosynthetic efficiency, comprising the steps of exposing the microorganisms grown in a culture tank of culture broth to light and carbon source; circulating the culture broth vertically from the bottom of the tank up to the surface of the tank to allow momentary exposure of the microorganisms to the light and carbon source, using one or more circulating means located within the culture tank which are regulated according to a selected cultivation mode; and maintaining the cultured microorganisms at a substantially constant areal density range of 300 to 12,000 g/m² to attain the optimal photosynthetic efficiency of the cultivation.

### FIELD OF INVENTION

The present invention relates to a method and a system for mass-cultivating photosynthetic microorganisms, especially microalgae, with enhanced photosynthetic efficiency. More particularly, the present invention relates to a method and a system for highly efficient capture of available energy from sunlight during outdoor mass-cultivation of microalgae, in which incoming sunlight energy can be optimally utilized for photosynthesis and thus preventing the occurrence of light saturation as well as photoinhibition during the culture's growth.

### BACKGROUND OF THE INVENTION

Microalgae are naturally found in freshwater and marine systems. They can exist individually, or in chains (filaments) or in groups (colonies) and represent an immense range of genetic diversity. Microalgae exhibit exceptional capabilities to replicate and grow. For nutritional requirements, they can derive their carbon autotrophically from carbon dioxide or bicarbonate, or heterotrophically from organic compounds. For energy, they can behave as phototrophs and use light as the energy source, or behave as chemotrophs to employ oxidation-reduction reactions to obtain energy. When both light and carbon source are provided, growth rate of certain species of microalgae under mixotrophic conditions could appear to be the sum of both. In addition, microalgae thrive in a wide range of pH as well as climatic conditions.

Research on mass-cultivation of microalgae has been conducted worldwide in various aspects. One of the major interests is centred on microalgae's apparent photosynthetic efficiency which approaches the theoretical optimal rates under low light conditions in the laboratory, while another is on the many useful products derived from microalgae. Different systems and apparatuses have also been designed and used for the cultivation of microalgae, ranging from natural unstirred ponds, circulated high rate ponds and photobioreactors which are designed to optimize light transfer to the microalgal crop. Majority of the cultivation is carried out in re-circulating high rate open pond. Due to high cost of installation and operation, photobioreactors are mainly used for experimental purposes or production of high value species where contamination is to be avoided. With the promise of being able to avoid contamination, heterotrophic fermentors have also been used, in which the microalgal crop can be fed with sugars.

In an outdoor algae cultivation system, where all major nutrients can be supplied to optimal amounts, sunlight becomes the limiting factor for photosynthetic growth and photosynthetic efficiency becomes the bottleneck. Potential photosynthetic efficiency can be estimated by the amount of incoming light suitable for photosynthesis, and the rate at which light energy can be converted into chemical energy. It is reported that only 45% of irradiation of sunlight falls within the photosynthetically active radiation (PAR) wavelength, which ranges from 400nm to 700nm. Applying the Stark-Einstein photochemical equivalence law to photosynthesis, the maximum rate of conversion of light energy into chemical energy is in the order of 27%. Therefore, the maximum photosynthetic efficiency of photosynthesis in sunlight is theoretically estimated to be generally in the order of 11.8% (25% of 47%).

However, actual photosynthetic efficiencies achieved with open ponds have been much lower. For example, during the Aquatic Species Program which was carried out under the auspices of National Renewable Energy Laboratory of the USA, average productivity at their Roswell Outdoor Test Facility (OTF) from 1987 to 1989 during the peak growing season from June through October was 19 g/m²/d and the year round average was 9.8 g/m²/d. However, very high productivities of 50 g/m²/d were observed on some occasions. In the desert areas of New Mexico where the OTF is located, the average daily insolation could be around 5.84 kilowatts, which translates to a potential productivity of 100 g/m²/d, thus illustrating the potential for improvement with open ponds. It is suggested that, the cause of the low photosynthetic efficiency is the presence of excess light, accounting for around 80% loss of efficiency through the light saturation and photoinhibition phenomena.

Microalgae have consistently shown photosynthetic efficiencies approaching the theoretical optimal values under low light and constant illumination conditions in the laboratory, at approximately 200 μmol photons m⁻² s⁻¹, and continue at high efficiencies until approximately 400 μmol photons m⁻² s⁻¹. However, once illuminated in higher light intensities, such as full sunlight which has an intensity of approximately 2,000 μmol photons m⁻² s⁻¹, there was a tendency for productivity to fall off by as much as 80%. Thus, laboratory experiments to determine optimal growth rates are conducted at low light levels.

The shortfall in photosynthetic efficiency has been explained by the light saturation and photoinhibition phenomena. In summary, light energy in the form of photons are captured at a speed in excess of the rate it can be utilized to synthesize carbon compounds. Excess energy is first dispersed and wasted by through heat and phosphorescence. This process is referred to as light saturation phenomenon. Further excess energy leads to damage to the photoreceptors, causing them to stop capturing energy. A period of rest, without excess photons, is required to return photoreceptors to a functional state. This phenomenon is referred to as photoinhibition.

The main activities of the OTF study appeared to be the determination of the water flow characteristics and operating parameters so that minimal energy was required to keep the algae in suspension for optimal utilisation of the incoming sunlight. There are three key features of the pond design and cultivation protocol which were validated at the OTF, including the light path should be as short as possible, the culture density (g/L) should be such that there is little or no mutual shading, and the horizontal motion is relied upon to keep the algae in suspension to minimise energy requirements. To meet all three requirements, pond depth was kept shallow around 30 cm. Recently published data show that culture areal densities, alternatively described interchangeably herein as “standing crop” measured as, weight of algal biomass per unit area, such as g/m², and productivity per unit area has not improved.

There are a few patented technologies over the prior arts relating to methods, systems and devices or apparatuses for cultivating microalgae. Some of the patented technologies disclose the methods or systems for enhancing the microalgae production by controlling limiting factors. Of interest in respect to a method for controlling cultivation of microalgae is Japanese Patent No. JP6000079(A), which deals with detection of the carbon dioxide concentration and keeping it within a specific acidic pH range. The efficiency of this method is controlled through the detection of bicarbonate. PCT publication No. WO2011035166(A1) discloses microalgae fermentation using controlled illumination, which is achieved by providing a light signal for improving heterotrophic growth. Another PCT publication No. WO2009134114 relates to an apparatus for mass-cultivation of microalgae and method for cultivating the same in which different types of cultivation modes can be selected in order to increase high yield of microalgae. However, the use of optimal areal density to capture available incoming sunlight which could avoid light saturation and photoinhibition has not been disclosed.

There are also several other patented technologies disclosed in the prior art relating to the prevention of photoinhibition in microalgae cultivation. For example, Japanese

Patent No. JP6292480(A) discloses a plant resistant to photoinhibition and its production, which is achieved by transforming a plant cell with an expression vector containing a DNA coding a glutamine synthetase and reproducing a plant body from the transplant. On the other hand, a method for enhancing cell productivity by reducing photosynthetic pigments from photosynthetic cells are disclosed in Korean Patent No. KR20040059182(A), in which the light-capturing capability is reduced, hence the photoinhibition is reduced, resulting in high productivity of cultivation. However, none of the patented technologies relates to the application of the innovative design or assembly considering areal culture density, or standing crop, in the methods or systems of microalgae cultivation, whereby its photosynthetic efficiency could be enhanced.

There has not been any patented technologies relating to an invention which can avoid light saturation and photoinhibition by adopting a specifically designed standing crop high enough to utilize the incoming light insolation, which also permits selection of the predominant cultivation mode, such as mixotrophic culture of microalgae that is capable of providing the highest productivity rate of microalgae. In order to overcome the drawbacks of the prior arts, it is desirable for the present invention to provide an improved method and system which permits cultivation of microalgae with high light-utilization efficiency. It is also desirable for the method or system to be able to facilitate the rapid mass-cultivation of the microalgae under selected nutrition modes, while retaining low costs of apparatus construction and operation.

### SUMMARY OF INVENTION

The primary object of the present invention is to provide an improved method and system for mass-cultivating microalgae which is capable of attaining a high photosynthetic efficiency through optimizing the areal density of the microalgae culture.

Another object of the present invention is to provide an innovatively designed method and system for mass-cultivating microalgae in which optimum incoming sunlight can be received and fully utilized by the microalgae culture thus preventing the occurrence of light saturation and photoinhibition that affect the microalgae culture's growth.

Still another object of the present invention is to provide a method and a system for cultivating microalgae which enables different types of cultivation modes, such as phototrophic, heterotrophic or mixotrophic, depending on the available sources of carbon and desired composition of the cultured microalgae.

Yet another object of the present invention is to provide a microalgae-cultivating system coupled with a microalgae-harvesting mechanism, whereby the separation of microalgae from its culture broth can be performed directly within the system, which is energy and cost-saving.

Still another object of the present invention is to provide a method and system for cultivating microalgae which is capable of achieving a higher biomass production over a period of time, thus giving rise of a wide range of valuable products from microalgae biomass such as human consumables or nutritional supplements, animal feeds and biofuels.

Further object of the present invention is to develop a system for cultivating microalgae which is capable of promoting reclamation of wastewaters thus conserving the environment.

Another further object of the present invention is to develop a method and system for cultivating microalgae massively which aids the carbon dioxide capture from large point emitters such as power plants, cement plants, steel plants, ethanol plants, petrochemical plants and others, thus helping in resolving environmental problems.

At least one of the preceding objects is met, in whole or in part, by the present invention, in which one of the embodiments of the present invention describes a method for cultivating photosynthetic microorganisms with optimal photosynthetic efficiency, comprising the steps of exposing the microorganisms grown in a culture tank of culture broth to light and carbon source; circulating the culture broth vertically from the bottom of the tank up to the surface of the tank to allow momentary exposure of the microorganisms to the light and carbon source, using one or more circulating means located within the culture tank which are regulated according to a selected cultivation mode; and maintaining the cultured microorganisms at a substantially constant areal density range of 300 to 12,000 g/m² to attain the optimal photosynthetic efficiency of the cultivation.

According to one of the preferred embodiments of the present invention, the optimal photosynthetic efficiency of the cultivation is estimated from potential photosynthetic growth, efficiency of energy capture and growth rate of the photosynthetic microorganisms cultured. Preferably, the photosynthetic microorganisms are microalgae.

According to another preferred embodiment, the present invention discloses a method for cultivating photosynthetic microorganisms with optimal photosynthetic efficiency which further comprises a step of inducing a horizontal circulation of the culture broth within dark zone of the culture tank to maintain the culture in substantially heterotrophic growth state.

Still another preferred embodiment of the present invention discloses a method for cultivating photosynthetic microorganisms with optimal photosynthetic efficiency which further comprises a step of feeding the cultured microorganisms with additional carbon sources and nutrients via one or more inlets according to the selected cultivation mode. Preferably, the cultivation mode is selectably authotrophic, substantially heterotrophic or mixotrophic.

Yet another preferred embodiment of the present invention discloses that the substantially constant areal density range of the cultured microorganisms are maintained by harvesting the photosynthetic microorganisms from the culture tank periodically.

According to yet another preferred embodiment of the present invention, the step of harvesting the photosynthetic microorganisms from the culture tank can be conducted by separating the photosynthetic microorganisms from the culture broth using a filtering means and collecting the photosynthetic microorganisms from a first outlet of the culture tank. Whilst, a step of recycling filtered water from a second outlet of the culture tank and a step of storing the filtered water can also be included according to another preferred embodiment of the present invention.

Further embodiment of the present invention is a method for cultivating photosynthetic microorganisms with optimal photosynthetic efficiency which further comprises a step of determining acidity of the culture broth for feed rate regulation. Preferably, the method can also include a step of determining light intensity in the culture tank for cultivation mode selection.

Another further embodiment of the present invention is a system for cultivating photosynthetic microorganisms with optimal photosynthetic efficiency, comprising a culture tank containing a culture broth grown with microorganisms which are exposed to light and carbon source; one or more circulating means located within the culture tank and regulated according to a selected cultivation mode, for circulating the culture broth vertically from the bottom of the tank up to the surface of the tank to allow momentary exposure of the microorganisms to the light and carbon source; wherein the cultured microorganisms are maintained at a substantially constant areal density range of 300 to 12,000 g/m² to attain optimal photosynthetic efficiency of the cultivation.

As set forth in the preceding embodiments, the photosynthetic microorganisms are microalgae. Preferably, the cultivation mode applied is selectably authotrophic, substantially heterotrophic or mixotrophic.

According to a preferred embodiment of the present invention, the circulating means of the system are airlift tubes of different lengths disposed at different positions within the culture tank. Preferably, the system further comprises a horizontal circulating means located within the culture tank for inducing a horizontal circulation of the culture broth within dark zone of the culture tank.

Another further embodiment of the present invention discloses a system for cultivating photosynthetic microorganisms with optimal photosynthetic efficiency which further comprises one or more inlets for feeding the cultured microorganisms with additional carbon sources and nutrients according to the selected cultivation mode. The inlets are preferably connected to a controlling means for controlling feed rate and sources of feed.

Still another further embodiment of the present invention discloses a system for cultivating photosynthetic microorganisms with optimal photosynthetic efficiency which further comprises a photosynthetic microorganisms-harvesting mechanism that includes a filtering means for separating the photosynthetic microorganisms from the culture broth, and a first outlet of the system for outputting the photosynthetic microorganisms. Preferably, this system also further comprises a filtered water recycling mechanism which includes a second outlet of the system for outputting filtered water from the filtering means, and a storing means for receiving the filtered water.

In accordance with yet another further embodiment of the present invention, the system for cultivating photosynthetic microorganisms with optimal photosynthetic efficiency further comprises a pH sensor for determining acidity of the culture broth; a light sensor for determining light intensity in the culture tank; and a means for covering the culture tank.

With the improved photosynthetic efficiency and in situ ability to condition the microalgae through denial of light energy or provision of carbon compounds, the present invention can be profitably utilized to capture energy from sunlight and also carbon dioxide from large emitters, whereby the products of microalgae cultivation can be used in the manufacture of a wide range of useful products including human food or nutritional supplements, animal feed as well as renewable biofuels.

Furthermore, the improved photosynthetic efficiency increases the oxygen supply within the culture tank and increases the potential rate of heterotrophic growth, which may otherwise be limited by oxygen transfer rate from the water surface.

By maintaining a high areal density (weight of crop being cultivated per unit area of surface area), the cultivated crop outcompetes the invading species for the limited available nutrients, thus minimizing the probability of invasion by weed algae, enabling low cost maintenance of the target species.

The open pond of the existing technologies is reported to be able to maintain a culture density of approximately 1,000 mg/1 and a depth of 30 cm, translating into an areal density of 300 g/m², but is unable to attain optimal photosynthetic efficiency. However, the method provided by the present invention works efficiently to provide an optimal photosynthetic efficiency in which the culture density can be maintained at approximately 1,000 g/m² for general purpose continuously fed photosynthetic algal culture, and may be reduced to 300 g/m² for photosynthetic algal cultures in exponential growth phase, or increased to 12,000 g/m² for senescent and mixotrophic algal cultures, with the upper limit set by rate of transfer of oxygen from the air into the culture medium. Therefore, higher yield of microalgal biomass can be obtained therethrough.

In addition, the present method and system can be utilized in wastewater treatment processes, and the treated water would be availed for reuse after further treatment as necessary, depending on its different usage. With the process intensification, that is to maintain a very high concentration of microorganisms in the culture broth, the present invention can also be profitably utilized in lieu of the existing technologies to provide secondary and tertiary treatment of wastewaters with lower power consumption than activated sludge systems.

One skilled in the art will readily appreciate that the present invention is well adapted to carry out the objects and obtain the ends and advantages mentioned, as well as those inherent therein. The embodiments described herein are not intended as limitations on the scope of the invention.

### DETAILED DESCRIPTION OF THE INVENTION

The present invention relates to a method and a system for mass-cultivating photosynthetic microorganisms, especially microalgae, with enhanced photosynthetic efficiency. More particularly, the present invention relates to a method and a system for highly efficient capture of available energy from sunlight during outdoor mass-cultivation of microalgae, in which incoming sunlight energy can be optimally utilized for photosynthesis and thus preventing the occurrence of light saturation as well as photoinhibition during the culture's growth.

Hereinafter, the invention shall be described according to the preferred embodiments of the present invention and by referring to the accompanying description. However, it is to be understood that limiting the description to the preferred embodiments of the invention is merely to facilitate discussion of the present invention and it is envisioned that those skilled in the art may devise various modifications without departing from the scope of the appended claim.

The present invention discloses a method for cultivating photosynthetic microorganisms with optimal photosynthetic efficiency, comprising the steps of exposing the microorganisms grown in a culture tank of culture broth to light and carbon source; circulating the culture broth vertically from the bottom of the tank up to the surface of the tank to allow momentary exposure of the microorganisms to the light and carbon source, using one or more circulating means located within the culture tank which are regulated according to a selected cultivation mode; and maintaining the cultured microorganisms at a substantially constant areal density range of 300 to 12,000 g/m² to attain the optimal photosynthetic efficiency of the cultivation.

In accordance with one of the preferred embodiments of the present invention, the photosynthetic microorganisms are microalgae. The microalgae cultured can be of any species, depending on the purpose and demand. This includes the fast-growing mixotrophic species, such as the Chlorella spp. for treatment of wastewaters, capture of carbon dioxide and production of renewable fuels, high value species such as the Arthrospira spp. (Spirulina) for health food production where the high culture density keeps invasion of weed algae to acceptable levels. However, the present invention does not limit the use of the system and method embodied herein for the cultivation of other types of algae.

According to the preferred embodiment, the present invention provides a cultivation protocol which achieves the optimal photosynthetic efficiency through an areal density (weight of biomass/area, e.g. g/m²) sufficient to receive incoming sunlight without suffering light saturation and photoinhibition. It can be a culture tank, a pond, or any other bioreactor with an air or liquid interface, with a means of vertical circulation where the photosynthetic microorganisms are brought to the interface with light, proceed through a photic zone of decreasing light intensity where photosynthesis may take place and then dark zone where there is insufficient light for photosynthesis. The method and system can include various vertical circulation means and nutrients feed systems to enable cultivation of the microalgae in the predominant nutrition or metabolic modes, depending on the available inputs and desired cellular composition of the microalgae product.

In accordance with the preferred embodiment of the present invention, the method for cultivating photosynthetic microorganisms with optimal photosynthetic efficiency is characterized by the application of areal concentration (weight of algae per unit of pond surface area upon which sunlight impinges on the culture broth, e.g. g/m²) of the microalgal cells, which is high enough so that all the sunlight shining on a particular area is fully utilized. To avoid light saturation and photoinhibition in an open system, which is caused by excess light energy, the algal population under a particular area of pond surface should ideally be able to utilize all the incoming energy for photosynthesis. Therefore, the present invention provides a method for microalgae cultivation with optimal photosynthetic efficiency which involves a step of maintaining the cultured microorganisms at a substantially constant areal density range of 300 to 12,000 g/m².

According to one of the preferred embodiments of the present invention, the optimal photosynthetic efficiency of the cultivation is estimated from potential photosynthetic growth, efficiency of energy capture and growth rate of the photosynthetic microorganisms cultured. Example 1 shows a process of determining the constant areal density of a microalgae culture which shall be maintained in order to attain the optimal photosynthetic efficiency of the cultivation. For instance, assuming that insolation of full sunlight at noon on a clear day is 1,000 watts/m²/hour on a horizontal plane, growth rate is 2% per hour, the optimal photosynthetic efficiency is 11%, energy content of the algae is 5.5kcal/g, the areal density or standing crop of the microalgae required to process the incoming light energy onto one square meter of pond surface is therefore approximately 988 grams.

In accordance with the preferred embodiment of the present invention, the starting point of experimentation would be an areal density of approximately 1,000 g/m², with the actual optimal areal density, volumetric culture density (g/L), depth (cm) and light dark fraction to be determined by experimentation with reference to, inter alia, maximum insolation, the photosynthetic microorganism or mix of microorganisms cultivated, their growth rates, light saturation point, as well as other factors. As 1,000 mg/L appears to be an upper limit to successful algal culture, which means that a depth of 98.8 cm is required to attain the standing crop of the estimated 988 g/m². With such high culture densities and depths, the algae must be circulated in the vertical plane to expose the algae to light at the culture tank surface.

According to the preferred embodiment of the present invention, vertical circulation is preferably achieved by utilizing airlift driven draught tubes to optimize light and gases utilization as well as byproduct disposal. Besides, the present invention also discloses a method for cultivating photosynthetic microorganisms with optimal photosynthetic efficiency to provide oxygen in the water column, which further comprises a step of inducing a horizontal circulation of the culture broth within dark zone of the culture tank, in order to exclude light from a substantial portion the algae cultured, and when fed with organic compounds, subject them to heterotrophic growth conditions which may increase the proportion of oil in their cell contents.

According to the preferred embodiment of the present invention, there are three types of cultivation modes which can be applied in the system and the cultivating method disclosed, which is phototrophic cultivation, heterotrophic cultivation or mixotrophic cultivation. Phototrophic cultivation or photoautotrophic cultivation allows the microalgae culture to obtain energy from light (sun) and carries out photosynthesis using inorganic simple materials, such as carbon dioxide, to generate energy for their growth and development. On the contrary, heterotrophic cultivation supplies the microalgae culture with organic compounds as their carbon sources as well as their chemical energy. Therefore, still another preferred embodiment of the present invention discloses a method for cultivating microalgae which further comprises a step of feeding the cultured microorganisms with additional carbon sources and nutrients via one or more inlets according to the selected cultivation mode, and circulating a substantial portion of the culture in the dark zone to exclude light energy and maintain them in heterotrophic growth mode. As an example, the culture broth of Chlorella spp. can be fed with organic compounds so that the microalgae can be grown heterotrophically in situ, with the intent of inducing a higher oil content.

For rapid biomass growth, the most preferred or the ultimate cultivation mode embodied in the present invention is the mixotrophic cultivation, whereby it allows the microalgae to derive metabolic energy both from photosynthesis and from external energy sources, such as organic waste materials present in or supplied into the culture tank.

Yet another preferred embodiment of the present invention discloses that the substantially constant areal density range of the cultured microorganisms are maintained by harvesting the photosynthetic microorganisms from the culture tank periodically. Under phototrophic cultivation mode, the areal density of the microalgae culture is preferably maintained at approximately 1,000 g/m². Representative volume of the microalgae cultivated can be withdrawn from the culture tank and weighed to determine its amount in mass, in order to obtain its density value. Calculation is then conducted to determine its areal density. Such process of sampling and determination can be conducted at selected intervals, such as every week, every month or every half a year.

According to yet another preferred embodiment of the present invention, the step of harvesting the photosynthetic microorganisms from the culture tank can be conducted by separating the photosynthetic microorganisms from the culture broth using a filtering means and collecting the photosynthetic microorganisms from a first outlet of the culture tank. Whilst, a step of recycling filtered water from a second outlet of the culture tank and a step of storing the filtered water can also be included according to another preferred embodiment of the present invention.

When the cultivated algae are harvested, all the inlets can be closed and the filtered water can be pumped out, leaving the algae isolated upstream of the filtering means. The microalgae can be removed by the outlet for microalgae, only leaving behind sufficient microalgae as seed for the subsequent cultivation process.

Further embodiment of the present invention is a method for cultivating photosynthetic microorganisms with optimal photosynthetic efficiency which further comprises a step of determining acidity of the culture broth for feed rate regulation.

Preferably, the method can also include a step of determining light intensity in the culture tank for cultivation mode selection.

Preferably, the cultivation mode can be altered by the operation of the plurality of inlets and the circulating means, as shown in Example 2. The cultivation mode selection is based on the light intensity obtained by the light sensor, or selected as the preferred mode due to the desirability of the algal contents when cultivated under the preferred mode. The reading of the pH sensor serves as a reference for the feedstock conditions and plays a role in the determination of the opening or closing of the feed inlets.

The cultivation modes disclosed by the present invention are innovative in various aspects. Above all, the circulating means operated in isolation of each other or in any combination avoids photoinhibition. In addition, the cultivation modes take advantage of the high light efficiencies of the microalgae in low light intensity environments as most of the time spent in the photic zone will be spent in a low light intensity regime. Besides, there are also avoidance of oxygen poisoning and improved gas exchange at all times as circulation brings the entire contents of the culture tank to the surface on a regular basis and enables the removal of excess dissolved oxygen during day time and removal of excess carbon dioxide during night time. Further, the circulating means also help in the self-flocculation of the culture making the microalgae easier to be harvested.

In a preferred embodiment, the apparatus and the method invented also enables a deeper culture zone, which is approximately up to 100 cm or more from the surface level of the culture broth; and by eliminating light saturation and photoinhibition, achieve a higher areal productivity approaching 155 g m⁻² d⁻¹ where daily insolation is 7,700 watts. As the culture density can be maintained in the order of 1,000 mg/L, invasion by weed algae can be controlled to maintain a monoculture. The incorporation of a pH sensor can also be used to keep pH values within a range favourable for algal growth, which is achieved by regulating the extent of vertical circulation and carbon dioxide supply so that air exchange is adequate to avoid CO₂ and oxygen build up. By using the heterotrophic growth mode, the residues of microalgae after extraction of the valuable products can be used as feedstock for the growing system. In this manner, almost all the carbon compounds can be extracted and turned into fuel, which is a significant contributor to overall energy efficiency.

Another further embodiment of the present invention is a system for cultivating photosynthetic microorganisms with optimal photosynthetic efficiency, comprising a culture tank containing a culture broth grown with microorganisms which are exposed to light and carbon source; one or more circulating means located within the culture tank and regulated according to a selected cultivation mode, for circulating the culture broth vertically from the bottom of the tank up to the surface of the tank to allow momentary exposure of each of the microorganisms to the light and carbon source; wherein the cultured microorganisms are maintained at a substantially constant areal density range of 300 to 12,000 g/m² to attain optimal photosynthetic efficiency of the cultivation.

In accordance with the preferred embodiment of the present invention, the system for microalgae cultivation disclosed can comprise a culture tank, which may be of any dimension as determined by the available space. However, an upper limit of 5,000 square meters with a central dividing wall taking the form of the well known high rate pond is recommended for use in the present invention, as it also allows wastewater treatment, capture of carbon dioxide as well as ease of operation management. For small scale operations, a lower limit of 2 meters wide and 10 meters long might be appropriate for cultivation of high value microalgae. The depth should not exceed 2 meters for safety reasons. In its larger form, the tank is essentially a pond and its walls and sides can be lined with impermeable material, optionally. In its smaller form, the tank can be made of steel, glass-reinforced plastics, cement, brickwork and others. As described in the preferred embodiment, the culture tank contains culture broth in which the microalgae culture is grown. This culture broth or culture medium should have a sufficient standing crop of photosynthetic micro-organisms to utilize the incoming sunlight. Preferably, the culture broth is supplied with carbon sources and nutrients via one or more inlets. The inlets can be a series of pipes and manifolds.

The microalgae cultivation system disclosed in the preferred embodiment of the present invention comprises one or more inlets connected to a controlling means which is capable of controlling the feed rate as well as the sources of feed supplied to the culture tank by opening and closing the inlets. Therefore, the carbon dioxide, organic carbon compounds and other nutrients can be inputted either via a single inlet, wherein the feedstocks are channeled together into the tank; or by multiple inlets, wherein the different types of feedstocks are channeled separately into the tank.

Preferably, three sets of inlets are assembled in the apparatus as disclosed in the preferred embodiment of the present invention. The first set of inlets is feed for carbon dioxide; the second set of inlets is feed for organic carbon compounds, such as domestic sewage, animal manures, leachate from landfills, sludge from sewage treatment plants, wastewater from food processing, vegetable oil including olive oil, palm oil and others, plants, blood from slaughterhouses, simple and complex sugars from the saccharification of plant biomass; and the third set of inlets is feed for other nutrients and trace elements, such as phosphorus, nitrogen, silica, calcium, magnesium, sodium, potassium, iron, manganese, sulphur, zinc, copper and cobalt in a balanced fashion to foster optimal growth. Where the incoming feed for organic compounds is such that it causes the growth of excessive bacterial matter in the tank, a portion or all of the feed may be passed through an anaerobic digester, prior to discharge into the tank. The carbon dioxide produced by anaerobic digestion is fed to the tank for phototrophic cultivation of the algae. Therefore, the culture medium generally comprises water, organic materials, dissolved oxygen, dissolved carbon dioxide, nutrients, trace elements and others.

Accordingly, the carbon source are carbon dioxide for photoautotrophic growth, the additional carbon source are organic carbon compounds for heterotrophic growth, and a combination of the two for mixotrophic growth. Preferably, other nutrients such as nitrogen, phosphorus, trace elements like silica, calcium, magnesium, sodium, potassium, iron, manganese, sulphur, zinc, copper and cobalt are added so that the limiting factors for growth shall only be the carbon supply, photoautotrophic light and oxygen transfer under high areal density (g/m²) conditions.

In practice, the optimal standing crop or areal intensity (g/m²) for the algal crop being cultivated must be determined by undue experimentation, with reference to tolerance to excess light energy before photoinhibition sets in, culture density (g/l), maximum insolation, growth rate, energy content of the algae and harvesting regime. As set forth in the preceding embodiments of the present invention, the cultivation mode applied can be authotrophic, heterotrophic or mixotrophic. However, when cultivated under mixotrophic conditions where organic materials provide a source of chemical energy, the upper limit of the areal density can be defined by the transfer rate of oxygen into the culture broth.

It is also known in the art that certain species of microalgae such as Chlorella spp. can be cultivated to modify their cell contents after a period of heterotrophic growth.

Thus, the use of one or more circulation means is vital. According to a preferred embodiment of the present invention, the circulating means responsible for the vertical circulation of the system are airlift tubes of different lengths disposed at different positions within the culture tank. These airlift tubes can be employed for different assemblies and purposes. According to the preferred embodiment, three different types of airlift tubes are used in the present invention. All airlift tubes can be assembled vertically to ease the circulation process.

In a preferred embodiment of the present invention, the first set of airlift tubes preferably begins slightly above the surface of the bottom of the tank, and terminates slightly lower than the level of the culture broth. It is a vertical circulation tube which projects the culture broth together with the microalgae so as to create a momentary exposure of the microalgae to high intensity, sunlight, thus avoiding photoinhibition. This circulation of the first set of airlift tubes can facilitate removal of any excess oxygen during daylight hours when photosynthesis takes place and removal of excess carbon dioxide during the night time. When the surface is exposed to photoautotrophic light, the microalgae will travel down to the bottom through the photic and dark zones due to the vertical mixing, experiencing a fluctuating light regime, or light and dark cycles.

The second set of airlift tubes begins slightly below the beginning level of the first set of airlift tubes and terminates slightly lower than the surface level of the culture broth. The circulation of the second set of airlift tubes is vital for transporting the culture medium, which microalgae has been separated therefrom, to replenish the culture tank. The second set of airlift tubes circulates the filtered water medium and assists to ensure that no anoxic pockets are formed throughout the culture tank.

The third set of airlift tubes begins approximately at the same beginning level of the first set of airlift tubes and terminates below the surface of culture broth, close to the commencement of the dark zone. This third set of airlift tubes are preferably coupled with a horizontal circulating means. For instance, it can be fitted with a deflecting means or deflector plates to reduce or eliminate the vertical velocity component and induce a horizontal velocity component to the upcoming culture broth so that the culture broth circulates within the dark zone. The reason for employing the third set of airlift tubes is to promote the heterotrophic cultivation of the microalgae in the dark. For instance, this cultivation mode is capable of decreasing the proportion of protein content of the microalgae Chlorella protothecoides and increasing its oil content to 55% or up to four times more than the fat content when cultivated by the phototrophic mode.

In the system disclosed in the present invention, only the upper surface of the culture broth is exposed to sunlight. The deeper parts of the culture broth get progressively darker and devoid of sufficient light energy for photosynthesis due to absorption by the microalgae, and mutual shading. The photic zone or light fraction is the zone where sufficient light is available to support photosynthetic activity. This light will be absorbed by the algal culture for photosynthesis. Depending on biomass concentration and absorption by algae present, there will be insufficient light to support photosynthesis beyond a certain depth. These deeper parts of the culture tank can be considered the dark zone, or dark fraction. Therefore, different types of vertical and horizontal circulating means are required to optimize the cultivation mode, production rate and culture density of microalgae.

An example of the different operation of inlets and airlift tubes are further detailed in Example 2. In accordance with the preferred embodiment of the present invention, the operation of the inlets and circulating means can be regulated to achieve the growth condition for different types of cultivation modes. During phototrophic or photoautotrophic growth of microalgae, both first and second sets of airlift tubes are operated and the culture broth is fed with carbon dioxide from the first set of inlets, with other nutrients and trace elements from the third set of inlets. Organic materials are not needed in this cultivation mode as the microalgae can derive energy from sunlight to carry out photosynthesis process. The nutrients and trace elements are added to remove growth limiting factors of the microalgae so as to achieve an optimum growth rate and culture density of the microalgae.

During the heterotrophic cultivation mode where photoautotrophic light is not needed, the first set of airlift tubes is not operated, while the second and third sets of airlift tubes are operated. In this manner, the bulk of the microalgae remain in the dark zone. The culture broth is fed with organic material, other nutrients and trace elements, but the carbon dioxide is withheld, therefore, only the second set and third set of inlets are opened during this cultivation mode. For mixotrophic growth, both first and second sets of airlift tubes are operated, and all three sets of inlets are opened to supply the culture broth with organic materials, carbon dioxide, other nutrients and trace elements.

Still another further embodiment of the present invention discloses a system for cultivating photosynthetic microorganisms with optimal photosynthetic efficiency which further comprises a photosynthetic microorganisms-harvesting mechanism that includes a filtering means for separating the photosynthetic microorganisms from the culture broth, and a first outlet of the system for outputting the photosynthetic microorganisms. Preferably, this system also further comprises a filtered water recycling mechanism which includes a second outlet of the system for outputting filtered water from the filtering means, and a storing means for receiving the filtered water. As set forth in the preceding description, the microalgae-harvesting process can be conducted periodically.

According to the preferred embodiment of the present invention, the storing means can be assembled at the bottom of the filtering means or be assembled externally. It can be another tank made of steel, glass reinforced plastics, cement, brickwork, or others, and comprises an outlet for water. The outlet for water is a means for discharging filtered water or filtered broth from the storing means. The dissolved organic materials in the incoming feed waters introduced through inlet pipes are substantially incorporated into the microalgae through its growth process and removed together with the microalgae and other matters from this filtered water or filtered broth. The filtered water can be sent for any suitable applications directly or to be further processed and sterilized for more hygienic purposes.

In accordance with another preferred embodiment of the present invention, the filtering means is a sand bed or a microscreen. A semi-permeable membrane can also be included for further filtering purposes. It is capable of separating the microalgae from the culture broth, and the microalgae is removed from the tank via the second outlet. Preferably, the outlet for microalgae is equipped with a pumping means externally or internally to assist the removal of microalgae.

Still another embodiment of the present invention is an apparatus for cultivating microorganisms, preferably microalgae, further comprising a pH sensor for determining acidity of the culture broth. One or more pH sensor can be assembled according to the dimension of the culture tank. The reading of acidity or alkalinity of the culture broth helps to regulate the feed rate of carbon source including carbon dioxide and organic carbon compounds. The pH of the entire culture broth can be modified by varying the supply of these carbon compounds, increasing the supply to decrease pH and vice versa.

In another embodiment of the present invention, the apparatus for cultivating microorganisms, preferably microalgae, further comprises a light sensor for determining light intensity in the culture tank. Preferably, one or more light sensor is assembled at predetermined depths of the culture medium to obtain readings of the light intensity. The readings are used to determine the time to initiate the removal of the cultivated microalgae, as well as to determine the time to switch the cultivation mode to heterotrophic condition.

Preferably, the apparatus according to the previous embodiments further comprises a means for covering the culture tank, wherein the means for covering is a photoautotrophic light transmitting material, for example polyethylene film. The means for covering is capable of preventing contamination by weed algae and reducing water and heat losses.

The method and system disclosed in the present invention can be employed to improve productivity and environmental sustainability in many aspects, including aquaculture, wastewater treatment, capture of carbon dioxide, production of human and animal food, control of eutrophication as well as production of renewable biofuels. With the system invented, an avenue is opened to produce low cost high protein feeds for animal husbandry. Should the combination of transparent cover and high culture densities be sufficient to maintain culture purity, food and nutritional supplements for human consumption can also be cultivated with a lower production cost.

Besides, the method and system as embodied herein may also be used as replacement for conventional activated sludge and pond systems presently deployed for waste water treatment. Organic waste bearing waters from various sources, for example, sewage, sewage sludge, animal manure, palm oil mill effluent, blood from slaughterhouses, and others can be inputted into the system, wherein the microalgae is grown, using up the dissolved organic materials, phosphates and nitrates. In this manner, tertiary standards of treatment and therebeyond can be achieved.

According to the preferred embodiment of the present invention, the algae thus grown should be used for renewable fuels due to the likely presence of pathogens. The oil fraction would be processed into fuels for compression ignition engines, the carbohydrate fraction fermented into ethanol or butanol, and the mash anaerobically digested into methane. The residues, containing phosphorus and residual nitrogen compounds, can be landfilled as fertilizer. Being freely scalable, the devise can be deployed at community level septic tanks, or for treatment of point sources of wastewater, such as wet markets.

At municipal solid waste landfill sites, food material may be separated from the incoming waste by washing to substantially remove organic materials prior to land filling, thus substantially avoiding the formation of leachate. The wash water is processed by the apparatus and recovered for reuse and the microalgae-removed for processing for biofuels. Where leachate already exists, the system can be used to treat the leachate prior to discharge into waterways.

As set forth in the foregoing description, the apparatus can be used to treat wastewater to tertiary standards prior to discharge into the waterways or subjected to further processing steps. This serves to stem inflow of new nutrients, and help with slowing down the environmental degradation. The nutrients accumulated in the waterways and found in the bottom layers of the apparatus can also be processed therein, where the mud is converted into the microalgae returning relatively clean and oxygenated water into the environment. The algae so produced should be processed into fuel in view of the likelihood of pathogens inhabiting the mud. In this way, the revenues generated can be used to fund the control of eutrophication and minimize its impact in the worst affected areas.

The present disclosure includes as contained in the appended claims, as well as that of the foregoing description. Although this invention has been described in its preferred form with a degree of particularity, it is understood that the present disclosure of the preferred form has been made only by way of example and that numerous changes in the details of construction and the combination and arrangements of parts may be resorted to without departing from the scope of the invention.

### EXAMPLE

Examples are provided below to illustrate different aspects and embodiments of the present invention. These examples are not intended in any way to limit the disclosed invention, which is limited only by the claims.

### Example 1

Case 1: During a study conducted from 29^{th} March to 13 Jun. 2011 at the CEHMM (Center of Excellence for Hazardous Material Management) near the Roswell facilities, the average daily pond productivity was 33.08 g/m²/d, with a maximum of 39.6 g/m²/d (25 Apr. 2011) and a minimum of 29 g/m²/d (29 Apr. 2011). Pond depth was kept at 12 inches (approximately 30 cm) and culture densities were reached as high as 1.2 g/1 (e.g., 29 Mar. 2011, 13 June 2011). As the algae responded negatively to overcrowding after a period of 24 hours, the ponds were harvested when densities reached >1 g/1 (39.5 g/m²). With culture densities capped at 1.0 g/L and pond depths of 30 cm, the maximum areal density would be 300 g/m².

Case II: It has also been reported that the Seambiotic facility at Ashkelon, Isreal, maintains a culture density of 1 g/L for the saline water species Nannochloropsis salina, and a depth of 20 cm, giving a culture density of 200 g/m². Sustained productivity was 20 g/m²/d.

Case III: At the 2,000 sq meter spirulina ponds in Musina, South Africa, the pond depth was 150mm, and culture densities reached 1.1 g/L. Thus, maximum areal density reached was around 165 g/m².

From the above, in view of the constraints imposed by the use of horizontal motion to effect mixing and the belief that the incoming light must penetrate to the bottom of the pond, the areal densities range from 165 g/m² to 300 g/m2. Under noon day sunlight conditions, and assuming a high rate of 2% in the increase in biomass concentration, about 897.84 g/m² of photosynthetic microalgae are required to utilize the incoming sunlight energy. This areal density varies inversely with growth rate; double if biomass concentration increases by only 1% per hour and halved biomass concentration increases by 4% per hour.

The standing crop of microalgae (g/m²) required to utilize the entire energy of the incoming overhead sunlight is computed in the following fashion:

TABLE 1
Minimum quantity of algae per sq meter
of pond to utilize peak insolation

| | | |
|---|---|---|
| Minimum angle above overhead | (a) | 0 |
| sun (degrees) | | |
| Cosine factor | (b) | 1 |
| Maximum insolation at noon (Solar | c = 1,000*(b)_Watts | 1,000 |
| insolation (1,000 watts) × | | |
| cosine of minimum latitude above | | |
| overhead sun) (Watts/hr/M2) | | |
| Maximum insolation at noon | d = c*3.63*1000_MJ | 3.64 |
| (MJ per hr/M2) | | |
| PAR % | e | 45% |
| Max Photosynthetic Efficiency | f | 25% |
| (Stark Einstein Theory −27%) | | |
| Potential energy capture (MJ) | g = (d) × (e) × (f) | 0.41 |
| k cal per MJ | h | 238.85 |
| Energy in algae (k cal/gm) | i | 5.50 |
| Potential photosynthetic growth | j = (g) × (h)/(i) | 17.78 |
| (gms/M2/hr) | | |
| Efficiency of energy capture | k | 0.90 |
| Growth rate | l | 2.00% |
| Required algal population | m = (j)/(k)/l | 987.84 |
| (gms/M2) to avoid photoinhibition | | |
| based on max insolation, per hour | | |

Under outdoor conditions, the actual amount of sunlight falling on a pond would depend on the angle of inclination of the light and cloud cover. Thus, outside of the first hours of daylight and late in the afternoon on a clear day, the areal densities of up to 300 mg/m² or so commonly utilised under present cultivation protocol cannot fully utilise the incoming energy, there being simply not enough microalgae present to process the high levels of insolation. Consequently, there will be excess light energy over that being photosynthesised, and the algae crop will experience light saturation followed by photoinhibition occurs ultimately resulting in a lack of photosynthetic efficiency.

To reduce the volume of culture broth to be circulated and also to be processed to harvest the algal crop it is desirable to maintain a high volumetric concentration (mg/L). However, the upper limit of volumetric density for rapid growth appears to be 1,000 mg/L before signs of overcrowding set in. To accommodate an areal density of 987.84 g/m², with this volumetric culture density, it would be necessary to have a pond depth of 98.7 cm.

At these volumetric culture densities, the photic zone (depth from the surface into which light will penetrate), is only in the region of 15 cm, leaving the remaining 85 cm dark. Thus, it is necessary to incorporate a means for vertical circulation to enable the entire algal crop to photosynthesise. The individual algal cell will experience a light regime of a light dark cycle, beginning with full insolation at the surface, gradually experiencing reducing intensity as it travels down the photic zone and complete darkness at the bottom of the pond. Assuming a pond depth of 100 cm and a photic zone of 15 cm the light dark cycle would be in the order of 15:85. The actual durations, culture volumetric culture densities (g/L) and depth (cm) would be determined by experiment with a view to optimising biomass growth and power required for circulation requirements.

### Example 2

The operation of inlets and circulating means according to different types of cultivation modes selected is shown in Table 2.

TABLE 2

| Component | Phototrophic | Heterotrophic | Mixotrophic |
|---|---|---|---|
| First set of Inlets | Open | Close | Open |
| Second set of Inlets | Close | Open | Open |
| Third set of Inlets | Open | Open | Open |
| First set of | On | Off | On |
| airlift tubes | | | |
| Second set of | On | On | On |
| airlift tubes | | | |
| Third set of | Off | On | Off |
| airlift tubes | | | |

1-23. (canceled)

24. A method for cultivating photosynthetic microorganisms with optimal photosynthetic efficiency, comprising the steps of:
estimating an optimal photosynthetic efficiency rate of the cultivation using data of potential photosynthetic growth, efficiency of energy capture and growth rate of the microorganisms cultured;
determining an areal density range of the microorganisms which is to be maintained in a culture tank, based on the optimal photosynthetic efficiency rate estimated;
exposing the culture tank containing the microorganisms grown in a culture broth at the predetermined areal density range to light and carbon source; and
circulating the culture broth vertically from the bottom of the tank up to the surface of the tank to allow momentary exposure of the microorganisms to the light and carbon source, using at least one circulating system located within the culture tank which are regulated according to a selected cultivation mode to attain the optimal photosynthetic efficiency of the cultivation.

25. The method according to claim 24, wherein the areal density range is maintained at a substantially constant rate of 300 to 12,000 g/m².

26. The method according to claim 24, wherein the photosynthetic microorganisms are microalgae.

27. The method according to claim 24, wherein the cultivation mode is authotrophic, heterotrophic or mixotrophic.

28. The method according to claim 24, further comprising a step of inducing a horizontal circulation of the culture broth within dark zone of the culture tank.

29. The method according to claim 24, further comprising a step of feeding the cultured microorganisms with additional carbon sources and nutrients via one or more inlets according to the selected cultivation mode.

30. The method according to claim 24, wherein the substantially constant areal density range of the cultured microorganisms are maintained by harvesting the photosynthetic microorganisms from the culture tank periodically.

31. The method according to claim 24, further comprising a step of harvesting the photosynthetic microorganisms from the culture tank by separating the photosynthetic microorganisms from the culture broth using a filtering system and collecting the photosynthetic microorganisms from a first outlet of the culture tank.

32. The method according to claim 24, further comprising a step of recycling filtered water from a second outlet of the culture tank and storing the filtered water.

33. The method according to claim 24, further comprising a step of determining acidity of the culture broth for feed rate regulation.

34. The method according to claim 24, further comprising a step of determining light intensity in the culture tank for cultivation mode selection.

35. A system for cultivating photosynthetic microorganisms with optimal photosynthetic efficiency, comprising:
a program for determining an areal density range of the microorganisms which is to be maintained in a culture tank, based on an optimal photosynthetic efficiency rate estimated using data of potential photosynthetic growth, efficiency of energy capture and growth rate of the microorganisms cultured;
a culture tank containing the microorganisms grown in a culture broth at the predetermined areal density range, which are exposed to light and carbon source; and
at least one circulating system located within the culture tank and regulated according to a selected cultivation mode, for circulating the culture broth vertically from the bottom of the tank up to the surface of the tank to allow momentary exposure of the microorganisms to the light and carbon source to attain the optimal photosynthetic efficiency of the cultivation.

36. The system according to claim 35, wherein the photosynthetic microorganisms are microalgae.

37. The system according to claim 35, wherein the cultivation mode is authotrophic, heterotrophic or mixotrophic.

38. The system according to claim 35, wherein the circulating system is airlift tubes of different lengths disposed at different positions within the culture tank.

39. The system according to claim 35, further comprising a horizontal circulating system located within the culture tank for inducing a horizontal circulation of the culture broth within dark zone of the culture tank.

40. The system according to claim 35, further comprising one or more inlets for feeding the cultured microorganisms with additional carbon sources and nutrients according to the selected cultivation mode.

41. The system according to claim 35, further comprising a controlling system connected to the inlets for controlling feed rate and sources of feed.

42. The system according to claim 35, further comprising a photosynthetic microorganisms-harvesting mechanism which includes a filtering system for separating the photosynthetic microorganisms from the culture broth, and a first outlet of the system for outputting the photosynthetic microorganisms.

43. The system according to claim 35, further comprising a filtered water recycling mechanism which includes a second outlet of the system for outputting filtered water from the filtering system, and a storing system for receiving the filtered water.

44. The system according to claim 35, further comprising a pH sensor for determining acidity of the culture broth.

45. The system according to claim 35, further comprising a light sensor for determining light intensity in the culture tank.

46. The system according to claim 35, further comprising a system for covering the culture tank.
